(19) United States
(12) Patent Application Publication
(10) Pub. No.: US 2016/0101048 A1
(43) Pub. Date: Apr. 14, 2016

(54) +L-CARNOSINE ZINC FORMULATIONS AND METHODS OF USE

(71) Applicants: Richard J. Di Rocco, Allentown, NJ (US); Dale M. Lombardi, Levittown, PA (US)

(72) Inventors: Richard J. Di Rocco, Allentown, NJ (US); Dale M. Lombardi, Levittown, PA (US)

(21) Appl. No.: 14/879,663

(22) Filed: Oct. 9, 2015

Related U.S. Application Data
(60) Provisional application No. 62/061,897, filed on Oct. 9, 2014.

(51) Int. Cl. A61K 9/00; A61K 9/06; A61K 47/36; A61K 38/05

(52) U.S. Cl. CPC A61K 9/0053; A61K 38/05; A61K 9/06; A61K 9/007; A61K 47/36

(57) ABSTRACT

Various formulations of L-carnosine zinc (L-CAZ), and methods of using and administering such formulations to patients suffering from various diseases, disorders, or conditions are disclosed. In particular, low average particle size L-carnosine zinc is employed in various pharmaceutical compositions, including aqueous matrices and hydrogels.

### CROSS REFERENCE TO RELATED APPLICATIONS

This application claims priority to U.S. Provisional Patent Application No. 62/061,897, filed Oct. 9, 2014, the entire contents of which are hereby incorporated by reference herein.

### FIELD

Disclosed herein are various formulations of L-carnosine zinc (L-CAZ), and methods of using and administering such formulations to patients suffering from various diseases, disorders, or conditions.

### BACKGROUND

N-(3-aminopropionyl)-L-histidinatozinc (L-Carnosine Zinc or L-CAZ) is a polymeric compound that has been shown to reduce inflammation and promote healing of ulcers of the gastrointestinal (GI) tract. L-carnosine zinc is registered and approved as an orally administered tablet or capsule for the treatment of peptic ulcers in Japan manufactured by Hamari Chemicals, Ltd. and sold by Zeria Pharmaceuticals, Ltd.

The synthesis, chemical characterization, and description of potential medical uses of L-carnosine zinc were described by Matsukura and Tanaka in Applicability of Zinc Complex of L-Carnosine [Biochemistry (Moscow) 65: 817-823, 2000]. L-carnosine zinc is a true chelate comprising L-carnosine, a naturally occurring dipeptide, and zinc that is manufactured in polymeric form of Formula I:

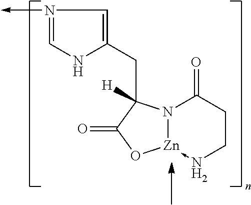

More and better formulations for the delivery of L-carnosine zinc are desirable to improve its effectiveness, applicability, to expand its usefulness, and for other reasons.

### SUMMARY

Some embodiments provide a pharmaceutical composition comprising L-carnosine zinc in an aqueous matrix of one or more pharmaceutically acceptable polymers. In some embodiments, the one or more pharmaceutically acceptable polymers are one or more linear or cross-linked polymers, such as those selected from but not limited to the group consisting of carbopol, polyacrylic acid, carboxymethylcellulose, hydroxyalkylcellulose, dextran sulfate, chitosan, and a water-soluble vinyl polymer, and combinations thereof. In some embodiments, xantham gum or similar materials may be used as the matrix material, alone or in conjunction with one or more other materials. In some embodiments, the L-Carnosine zinc comprises a plurality of particles having an average particle size (also referred to as average diameter or mean diameter) between about 0.5 micron and about 2 microns. In some embodiments, the pharmaceutical composition is a hydrogel. In some embodiments, the composition has a viscosity suitable for oropharyngeal lavage. In some embodiments, the composition has a viscosity suitable for swallowing.

Some embodiments provide a method of treating gastrointestinal and other body-organ system mucopathies including administering a composition described herein to a patient in need thereof. Some embodiments provide a method of treating the gastric mucosa, comprising administering a composition comprising a plurality of L-Carnosine zinc particles in an aqueous matrix directly to a wound site on the gastric mucosa. In some embodiments, the administration includes applying the composition via an endoscope.

Some embodiments provide a method of treating the oropharyngeal mucosa, comprising administering a composition comprising a plurality of L-Carnosine zinc particles in an aqueous matrix directly to a wound site on the gastric mucosa. In some embodiments, the composition has a viscosity suitable for lavage. In some embodiments, the administration comprises contacting the oropharyngeal mucosa by lavage with the composition. In some embodiments, the patient is permitted to expectorate any remaining composition after the lavage step. In some embodiments, the lavage is conducted for about 0.5 to about 5 minutes.

Some embodiments provide a method of treating disorders of the upper and lower gastrointestinal tract. Such methods comprise contacting the upper gastrointestinal tract of a patient in need of such treatment with a composition comprising a plurality of L-Carnosine zinc particles in an aqueous matrix. In some embodiments, the composition has a viscosity suitable for swallowing. In some embodiments, the contacting is facilitated by swallowing the composition. In some embodiments, the contacting is facilitated by endoscopic application of the composition applied directly to or in the proximity of a mucosal ulcer or site of inflammation.

Some embodiments provide a method for treating a wound site in a mucosa, comprising causing simultaneous anti-oxidant activity of the zinc moiety and the carbonyl trapping activity of the L-carnosine moiety within mutual intra-molecular atomic scale proximity to a site of mucopathy by administering a composition comprising a plurality of L-Carnosine zinc particles in an aqueous matrix.

In some embodiments, L-carnosine zinc is dissolved in a suitable solvent for administration to a human such as a suitable lipid. In some embodiments, the lipid solution of L-carnosine zinc is encapsulated in a softgel capsule.

Other embodiments will be apparent upon reading the following specification. This description is meant to be illustrative in nature. Many obvious variants will be apparent to one of ordinary skill in the art without departing from the spirit and scope of this disclosure.

### DETAILED DESCRIPTION

The effectiveness of L-carnosine zinc is attributed, in part, to its ability to adhere to mucosa of the GI tract to provide a beneficial coating to ulcers of gut mucosa; to inhibit inflammation signaling pathways (for example, suppression of IL-8 secretion induced by TNF-α or IL-1β in a dose-dependent manner, to down-regulate NF-κB and IL-8 expression); to promote wound healing (for example, by stimulating cell proliferation and migration); to inhibit H. pylori colony growth in the stomach (for example by inactivating urease, which H. pylori requires to support growth in acidic conditions); to protect tight junctions between intestinal epithelial cells by inhibiting apoptosis in these cells after a variety of insults related to chemotoxicity, radiation exposure, hypoxia and reperfusion injury; to inhibit increased gut permeability and/or gastropathies caused by exposure to high dose non-steroidal anti-inflammatory drugs (NSAIDS); to stimulate production of HSP72 a Heat-Shock Protein that has been shown to protect mucosal and other cell types. In addition to its own healing properties, L-carnosine zinc dissociates in the acidic environment of the stomach to at least partially release Zinc, which provides additional healing effect and to release L-carnosine, which provides both antacid and carbonyl trapping activity

Collectively, these attributes confer on L-carnosine zinc a unique constellation of muco-adhesive and gastro-protective and healing properties. These properties allow L-carnosine zinc to be an effective treatment and preventive for ulcerative and inflammatory disorders along the entire length of the GI tract. We propose enhancing these effects and expanding the use of L-carnosine zinc by providing new formulations of L-carnosine zinc.

Of special interest is the finding that administration of L-carnosine zinc is more effective than the co-administration of a zinc salt together with L-carnosine in a simple mixture. Matsukura and Tanaka in Applicability of Zinc Complex of L-Carnosine [Biochemistry (Moscow) 65: 817-823, 2000], suggest that this results from the specific adherence and direct delivery of L-carnosine zinc into the ulcer tissue when the chelate is used, whereas the therapeutic effect of a mixture of zinc salt and L-carnosine is diminished by a larger distribution volume in the whole stomach.

Without wishing to be bound by the theory, it is believed one reason for the greater therapeutic effect of L-carnosine zinc over the simultaneous administration of a mixture consisting of a zinc salt and L-carnosine is that the therapeutic benefit of the L-carnosine zinc is related to its molecular structure. L-carnosine zinc's unique structure provides a mechanism for the simultaneous delivery of a carbonyl trapping moiety, L-carnosine, and an anti-oxidant moiety, zinc, in mutual Angstrom scale proximity, when applied to a pathogenic site in a patient suffering from one or more of the pathologies described herein.

In addition to these therapeutic activities of the chelate, L-carnosine zinc contains the two moieties, cited above, and the molecular structure of the chelate serves as a medical device and/or pro-drug system for the delivery of the L-carnosine moiety, which then acts as a carbonyl trapping agent, and the zinc moiety which then acts as an anti-oxidant within mutual Angstrom scale proximity. Previous studies have determined that L-carnosine acts by chemically binding to, and sequestering, the aldehyde and/or ketone products of lipid peroxidation (A. R. Hipkiss, et al. Ann. N.Y. Acad. Sci. 959: 285-294 (2002). Ann. N.Y. Acad. Sci. 959: 285-294). Increased levels of lipid peroxidation have been repeatedly demonstrated as a part of the non-enzymatic “inflammatory cascade” process that underlies the secondary etiology of chronic inflammatory diseases. The various in vivo sources of toxic aldehydes have been reviewed by O'Brien et al. (2005). Previous studies have also determined that zinc acts as an anti-oxidant to neutralize reactive oxygen species (ROS) that are generated by oxidative stress. The anti-oxidant activity of zinc is thought to result from the reduction of hydroxyl radical formation from hydrogen peroxide through the antagonism of redox-active transition metals, such as iron and copper. (S. R. Powell, 2002). In an active pathogenic site in a body tissue, ROS and toxic aldehydes are generated in parallel in mutual Angstrom scale proximity. The ability of L-carnosine zinc to provide a system for the delivery of L-carnosine to trap toxic aldehydes and zinc to trap ROS in tandem and at mutual atomic scale proximity stoichiometrically eliminates both toxic aldehydes and ROS. The simultaneous temporal, and Angstrom scale proximity, of these actions may interfere with the synergistic aspects of toxic aldehydes and ROS in a manner that is not duplicated by administration of a mixture of a zinc salt together with L-carnosine.

By reducing polymeric L-carnosine zinc particle size, exposure of the zinc and L-carnosine moieties, in Angstrom scale proximity, to a therapeutic site of action can be increased due to an increase in the ratio of surface area to particle size. Lower L-carnosine zinc average particle size also advantageously reduces or eliminates settling of L-carnosine zinc particles when they are suspended in a hydrogel formulation or in an aqueous matrix solution. These properties permit greater contact between L-carnosine zinc and the wound site than current formulations and provide more effective therapeutic treatment and efficacy. The use of the compositions and methods described herein provide, among other things, enhanced exposure of the L-carnosine zinc complex and, to the extent it dissociates, exposure of each of the therapeutic moieties. Enhanced exposure of the chelate, as well as its therapeutic moieties upon dissociation, will therefore provide an enhanced therapeutic benefit for the promotion of healing of inflammatory and/or erosive mucopathies in mucosal tissues. Exemplary mucopathies include but are not limited to stomatitis, mucositis, Bechet's disease, aphthous ulcer, bullous pemphigoid, chemical cystitis, radiation cystitis, erythema multiforme, esophagitis, eosinophilic esopahagitis, peptic ulcer, Crohn's Disease; ulcerative colitis, radiation proctitis, interstitial cystitis, oral Lichen planus, or pemphigus. Some embodiments are particularly well-suited for the promotion of healing inflammatory and erosive mucopathies, particularly in any of the gastrointestinal mucosal tissues.

Reduced Particle Size

Some embodiments comprise L-carnosine zinc compositions having a plurality of L-carnosine zinc particles having a reduced particle size. Reduced particle size is believed to increase the surface area and thus increase the contact between the L-carnosine zinc and a wound site or site of inflammation. Current L-carnosine zinc formulations have an average particle size greater than about 2 microns. It is contemplated herein that average particle sizes be reduced to a maximum suitable to reduce granularity, and to minimize settling of L-carnosine zinc in various hydrogel suspensions disclosed herein. In some embodiments, the average particle size is less than about 2 microns. In some embodiments, the average particle size will be about 0.1 micron, about 0.2 micron, about 0.3 micron, about 0.5 micron, about 0.75 micron, about 1 micron, about 1.25 microns, about 1.5 microns, about 1.75 microns, or about 2 microns. In some embodiments, the average particle size is about 0.1 micron to about 2 microns; about 0.5 micron to about 1.5 microns; about 1 micron to about 2 microns. In some embodiments, the average particle size is any number or range of numbers between any two numbers recited above. In some embodiments, the particle size may be reduced to nanoparticle sizes (that is below about 0.1 micron).

In some embodiments, particle distribution is relatively narrow. In some embodiments, 50% or more of the particles will be within 20% of the average particle size. In some embodiments, 50% or more of the particles will be within 10% of the average particle size.

In some embodiments, a plurality of L-carnosine zinc particles, with an average particle size less than 2 microns or as otherwise described above, will have an average particle size distribution such that 90% of particles are less than 7 microns, less than 50% of particles are less than 2 microns, and less than 10% of particles are less than 0.5 microns. In some embodiments, about 50% of particles are less than about 1.0-2.0 microns, and 90% of particles are less than 5.0-7.0 microns. In some embodiments, about 50% of particles are less than about 1.0 micron, and 90% of particles are less than 5.0 microns. In some embodiments, about 50% of particles are less than about 2.0 microns, and 90% of particles are less than 7.0 microns. In some embodiments, about 50% of the particles are less than about 1.0 micron and 90% of the particles are less than about 5 microns. In some embodiments, about 50% of all particles are less than about 1 micron. In some embodiments, about 90% of all particles are less than 5.0 microns.

The average particle size of the L-carnosine zinc can be reduced through known micronization techniques. In some embodiments, the micronization leads not only to smaller average particle sizes, but also more uniform distribution, particularly at the lower end of the micron size range.

Viscous Formulations

In some embodiments, a composition is formed of a plurality of L-carnosine zinc particles and an aqueous matrix. The combination of L-carnosine zinc and the aqueous matrix is prepared in such a way that the viscosity of the composition can be controlled. Depending on the desired use, the viscosity can be about 3 to about 10,000 centipoise (cps). The viscosity can be varied depending upon the end use. For example, a relatively high viscosity is useful for adhering the composition to a wound site, a relatively low viscosity is useful for compositions which are to be swallowed, and a middle range viscosity is useful for lavage.

The viscosity may be relatively high, for example about 5000 cps to about 10000 cps, such that the composition can form a hydrogel or similar composition which may be applied and adhered directly to a wound site.

In other embodiments, the viscosity may be relatively low, for example about 3 cps to about 5000 cps, to facilitate swallowing but thick enough to facilitate coating of the esophagus or adherence to lesions, premalignant or hyperplastic conditions such as Barrett's Esophagus, or other inflammatory disorders, lacerations or ulcers in the esophagus. Some embodiments contemplate about 1000 cps. Formulations developed for swallowing may be used to coat the mouth, esophagus, and even the lining of the stomach. This is particularly useful for treatment of peptic ulcers, and as a component of a combination treatment in stomach cancer patients for eradicating H. pylori.

In the middle is a moderately viscous formulation that permits lavage in the mouth allowing L-carnosine zinc to be delivered to and adhered to lesions, and other wounds and pathologies in the mouth and throat, with the remainder being expelled. Viscosities of about 3000 cps to about 7000 cps are suitable for lavage formulations.

The reduced-particle size L-carnosine zinc can be present in liquids of varying viscosities suitable for the specific requirements necessitated by administration via direct application, lavage, or for swallowing. In some instances, the viscosity is about 3 to about 10000 cps. In some embodiments, the viscosity is about 5000 cps to about 10000 cps. In some embodiments, the viscosity is about 3 cps to about 5000 cps. In some instances, the viscosity is about 3000 cps to about 7000 cps. In some instances, the viscosity is about 3 cps, about 5 cps, about 10 cps, about 50 cps, about 100 cps, about 500 cps, about 1000 cps, about 1500 cps, about 2000 cps, about 2500 cps, about 3000 cps, about 3500 cps, about 4000 cps, about 5000 cps, about 5500 cps, about 6000 cps, about 7000 cps, about 7500 cps, about 8000 cps, about 8500 cps, about 9000 cps, about 9500 cps, about 10000 cps, or any number or range of numbers between any two numbers recited above.

The aqueous matrix may be any such matrix. An aqueous matrix of one or more additional mucoadhesive polymers is particularly useful. In some embodiments, the one or more mucoadhesive polymers include, but are not limited to, one or more linear or cross-linked polymer(s) selected from the group consisting of polyacrylic acid, carbopol, carboxymethylcellulose, hydroxyalkylcellulose, dextran sulfate, chitosan, a water-soluble vinyl polymer, xanthum gum and combinations thereof.

Compositions comprising a plurality of L-carnosine zinc particles and an aqueous matrix may contain about 3% to about 25% L-carnosine zinc particles by weight. In some embodiments, the L-carnosine zinc particles will be present at about 3%, about 4%, about 5%, about 7%, about 10%, about 15%, about 20%, or about 25%. In some embodiments, the L-carnosine zinc particles are present at any value or range of values between any two of the above recited values. The amount of L-carnosine zinc may exceed about 25% by weight of the composition, particularly in the more viscous hydrogel formulations. In particular, L-carnosine zinc may be present at about 25% to about 90% by weight. In some embodiments, the L-carnosine zinc may be present at about 30%, about 40%, about 50%, about 60%, about 70%, about 80%, about 90% or any value or range of values between any two of the above recited values.

For direct application, hydrogel compositions may be used. Any pharmaceutically acceptable hydrogel may be used, including, but not limited to those made from the materials listed above. In a hydrogel composition, the polymeric L-carnosine zinc particles will be suspended with the polymeric hydrogel to form a polymeric composite material for direct or substantially direct contact with the wound site. The hydrogel suspension of L-carnosine zinc can be administered directly through an endoscope to an ulcer in the upper or lower GI tract.

The composition is suitable for use at any mucosal site, including those of the digestive system such as the oropharyngeal, esophageal, gastric, intestinal, rectal, anal or other gastrointestinal mucosa, but also the nasal mucosa, genital mucosa, and urethral mucosa. As a hydrogel, the composition has gel-like viscosities suitable for positioning and maintaining positioning on the mucosa.

In some embodiments, the composition forms a pseudo-plastic liquid which facilitates application and adherence. In some embodiments, the L-carnosine zinc composition for direct application may be formulated as a viscous hydrogel suspension, or as a lipid based cream, paste, ointment or solution.

In other embodiments, the L-carnosine zinc particles are suspended in a less viscous aqueous matrix or solution. With appropriate selection of L-carnosine zinc particle size, and sufficient viscosity of the matrix, settling can be minimized or eliminated. With the smaller particle sizes discussed above, settling can be limited, which is important from a marketing perspective, but also facilitates uniform distribution of particles at the administration site and enhanced efficacy. Appropriate viscosities for lavage and for swallowing can be made by adjusting either or both the L-carnosine zinc and the polymer concentrations in the aqueous matrix. In the case of lavage, an appropriate viscosity composition is provided to a patient for swishing and/or gargling in the mouth and throat followed by expectoration. During this process, the L-carnosine zinc particles are brought into intimate contact with any wound sites, such as lesions, etc., within the oral cavity, throat, and oropharynx region. Due to its muco-adhesive properties, a quantity of L-carnosine zinc particles will attach themselves at any wound site present. It is contemplated that L-carnosine zinc, by virtue of its mucoadhesive properties, as well as those of the aqueous matrix will also be coated with L-carnosine zinc particles. Any L-carnosine zinc and aqueous matrix that is not adhered to a lesion or site of inflammation can be expectorated from the mouth, with the residual remaining in contact thereto. In this manner, L-carnosine zinc particles are provided and remain in contact with a wound site within the oral cavity. The viscosities can be altered to make similar application for treating the colon, or for making a cream, ointment or hydrogel for treating the rectum, anus, or vagina.

In other embodiments, compositions comprise a hydrogel suspension of L-carnosine zinc that can be delivered orally by swallowing to treat or prevent inflammatory and ulcerative conditions of the upper GI tract. The viscosity of such a composition is such that it is fluid and able to be swallowed, but thick enough to coat the upper GI tract. Administration of this type of formulation is easily accomplished by swallowing the solution. Due to its viscosity, the hydrogel suspension will have a coating action, clinging to the sides of the esophagus as it works its way through the GI tract. Along the way, as the solution finds and coats various wound sites, the L-carnosine zinc particles will, due to their mucoadhesive properties aided by the aqueous matrix, adhere to and remain at the wound sites encountered along the GI tract. Excess L-carnosine zinc and matrix will pass through the remainder of the GI tract. In this manner, L-carnosine zinc can be administered orally, but reach wound sites further within the GI tract, without the need for surgery or use of an endoscopic or other invasive procedure.

Through the use of various and appropriate adjuncts, L-carnosine zinc, particularly small particle size L-carnosine zinc, can be formulated into solution in a lipid cream, ointment or paste containing L-carnosine zinc alone or in combination with other medicaments for direct application to ano-rectal tissue, so as to treat inflammation such as anal fissures, hemorrhoids, and pruritis ani or other ano-rectal pathologies as well as vaginal pathologies.

Those of skill in the art will recognize that additional excipients, such as fillers, buffers, sweeteners, solubilizers, viscosity enhancers, flavorings, preservatives, and the like may be employed in various amounts and combination to achieve the desired effect.

In various embodiments, the L-Carnosine zinc, having the features described herein, such as small particle size and/or low molecular number, can be incorporated into various dosage forms, including liquids, hydrogels, tablets, tablet within a tablet, creams, capsule, gel-capsule, ointments, suspensions, syrup, elixir, nasal sprays, aqueous solutions, non-aqueous solutions, etc. The dosage forms may be unit dosage forms, extended or delayed release forms, or any other suitable dosage form.

In some embodiments, the L-carnosine zinc formulation can be adjusted with the addition of buffers, sugars, and the like to prepare an isotonic or isomolar composition compared to body fluids, such as but not limited to blood, lymph or interstitial fluid. For example, hydrogel suspensions of L-carnosine zinc can be prepared with one or more preservatives, such as sodium benzoate. Various concentrations of sweeteners such as sucrose, glucose, dextrose, other natural sweeteners, artificial sweeteners, etc. can be added to enhance the taste. Artificial or natural flavors and flavorants such as citrus, anise, mint, and other flavorings can be used in an effort to create the optimal flavoring of the hydrogel suspension of L-carnosine zinc. Additional fillers, stabilizers, and other excipients may be used to achieve desired characteristics. The overall osmolality of the preparation is preferably as close as possible to human body fluid to avoid hypertonic or hypotonic stimulation of nerve endings in open ulcers or other types of wound or inflamed tissues.

Low average particle size L-carnosine zinc can also be solvated in various lipids and emollients as required to form cream or ointment formulations useful for topical applications to wounds, abrasions and inflamed tissues throughout the body.

Methods of Treatment

As alluded to above, small average particle size L-carnosine zinc can be administered to a mucosal wound site by various methods, for various wound sites or areas of inflammation, ulceration, laceration, fissure or areas of pathogenic processes that lead to increased inter-epithelial permeability or epithelial cell apoptosis or necrosis.

In some embodiments, a method of treating gastric wound sites, such as but not limited to gastric ulcers, includes applying a composition comprising L-carnosine zinc and an aqueous matrix directly to the wound site. In some embodiments, the composition is applied via an endoscopic instrument. In some embodiments, the composition is a relatively high viscosity composition of L-carnosine zinc and an aqueous matrix, as described above. Small particle size L-carnosine zinc as described above are used to facilitate contact between the L-carnosine zinc and the wound site.

Endoscopic delivery of L-carnosine zinc in a highly viscous, cream, paste hydrogel or other formulation to lesions may occur anywhere within the GI tract that can be accessed endoscopically. Direct endoscopic application is an important new use that has potential to change the way GI ulcers are treated in hospital and outpatient care settings. In the case of bleeding ulcers, for example, after electro-cautery to stop the bleeding, the physician can endoscopically apply a coating of a viscous L-carnosine zinc formulation directly to the wound to promote healing. The same procedure can be used to treat bleeding ulcers elsewhere in the GI tract, such as those that occur in the colon in the context of inflammatory bowel disease, Crohn's disease, radiation proctitis, inflammatory and ulcerative complications of C difficile and other opportunistic infections of the lower bowel. Direct endoscopic application of L-carnosine zinc can also be employed for non-bleeding but serious ulcers in the esophagus, stomach, colon, and other mucosa.

Methods of treating wound sites in the oropharynx region comprise administering a composition comprising small particle size L-carnosine zinc in an aqueous matrix, as described above, by providing the composition to a patient in need of such treatment, and allowing them to wash the oral cavity and throat area with the composition, and expectorating any remaining fluid. In some embodiments, the wash constitutes swishing and/or gargling to facilitate good contact with the entire area. The wash can last from 30 seconds to 5 minutes. In some embodiments, the wash lasts about 30 seconds, about 1 minute, about 1.5 minutes, about 2 minutes, about 3 minutes, about 4 minutes, about 5 minutes. In some embodiments, the amount of the L-carnosine zinc composition used may be sufficient to substantially fill the oral cavity to reduce the need to move solution around the cavity for good coverage.

Methods of treating wound sites present in the upper GI tract include administering a composition comprising small particle size L-carnosine zinc in an aqueous matrix, as described above, to a patient in need of such treatment by providing the composition to the patient, allowing the patient to swallow the composition. Swallowing facilitates delivery of the composition to the walls of the upper GI tract, where the L-carnosine zinc associates with and adheres to one or more wound site.

In some embodiments, L-Carnosine zinc formulations may be administered for release in the stomach as treatment in stomach cancer patients to enhance eradication of H. pylori infection of gastroesophageal tissues in such patients. The mucoadhesive nature of L-carnosine zinc, particularly when presented as a hydrogel, localizes the L-carnosine zinc on the gastroesophageal mucosa and places the medicament where it is needed in these cancer patients for the eradication of H. pylori.

Some embodiments include the application of L-Carnosine zinc-containing hydrogels, pastes, creams, and ointments to external surfaces (i.e., skin) to promote the healing of wounds, such as bed sores and diabetic wounds, cuts and abrasions, and anal fissures.

L-carnosine zinc may also be incorporated into various lipid solutions for any of the above uses. as well as other uses.

### EXAMPLES

### Comparative Example 1

### Preparation of Xanthum Gum Hydrogel Suspension of L-Carnosine Zinc

The following ingredients were prepared as described below:

Water

Xanthum gum (0.75%)

Polymeric L-carnosine zinc (150 mg/5 ml final composition volume)

Anise extract (flavorant)

Step 1) A hydrogel consisting of xanthum gum (0.75%) in distilled water was prepared by mixing Xanthum gum in distilled water at room temperature under one atmosphere of pressure

Step 2) Polymeric L-carnosine zinc with a particle size range of about 0.1 micron to about 85 microns, with a particle distribution such that 90% of the particles are 24.925 microns or smaller, 50% are 5.664 microns or smaller, and 10% are 0.826 or smaller, and the mean diameter was 9.878 microns was added to the hydrogel prepared in Step 1 to produce a composite polymeric hydrogel consisting of 150 mg L-carnosine zinc suspended in 5 ml of hydrogel. The preparation was almost tasteless.

Step 3) Anise extract added in an amount sufficient to provide a mildly pleasant taste.

Over a period of 24 hours, the larger suspended particles settled slowly to form a thin layer of particulate L-carnosine zinc at the bottom of the container in which the hydrogel suspension was stored.

### Example 1

### Hydrogel Containing Small Particle Size L-Carnosine Zinc

A hydrogel suitable for direct administration to a gastric ulcer includes:

about 3% to about 25% L-carnosine zinc particles having an average particle size of about 0.5 micron to about 2 microns; and

an aqueous suspension containing one or more pharmaceutically acceptable polymers.

### Example 1A

### Preparation of Xanthum Gum Hydrogel of Example 1

The following ingredients were prepared as described below:

Water

Xanthum gum (0.75%)

low average particle size polymeric L-carnosine zinc (150 mg/5 ml final composition volume)

Step 1) preparing a hydrogel by mixing xanthum gum (0.75%) in distilled water at room temperature under one atmosphere of pressure to yield a xanthum based hydrogel;

Step 2) adding low average particle size polymeric L-carnosine zinc having average particle size less than about 2 microns to the xanthum based hydrogel from Step 1 to produce a composite polymeric hydrogel consisting of 150 mg low average particle size L-carnosine zinc suspended in 5 ml of hydrogel.

Optional flavorants, sweeteners, preservatives and other excipients may be added as desired.

Viscosity can be varied within a wide range, such as from 3 centapoise (=milk) to 5000 centapoise (=Karo syrup), by varying the amount of xanthum gum alone or in conjunction with filler or other excipient such as cellulosic material. For further reference, Castor oil is 1000 centapoise. It is anticipated that a more viscous hydrogel suspension of L-carnosine zinc will be optimal for direct endoscopic application to gastrointestinal ulcers.

### Example 2

### Lavage Formulation

A composition suitable for lavage application comprises:

about 3% to about 25% L-carnosine zinc particles having an average particle size of about 0.5 micron to about 2 microns; and

an aqueous suspension containing one or more pharmaceutically acceptable polymer; having a viscosity sufficient to facilitate lavage.

### Example 3

### Formulation Suitable for Administration Via Swallowing

A composition suitable for swallowing comprising:

about 3% to about 25% L-carnosine zinc particles having an average particle size of about 0.5 micron to about 2 microns; and

an aqueous solution containing one or more pharmaceutically acceptable polymer; having a viscosity sufficient to facilitate lavage.

What is claimed is:

1. A pharmaceutical composition comprising:
L-carnosine zinc in an aqueous matrix of one or more pharmaceutically acceptable polymers;
wherein the L-carnosine zinc is present at an average particle size of about 0.1μ, to about 2μ.

2. The pharmaceutical composition of claim 1, wherein the one or more pharmaceutically acceptable polymers are one or more linear or cross-linked polymers.

3. The pharmaceutical composition of claim 1, wherein the one or more pharmaceutically acceptable polymers is selected from the group consisting of polyacrylic acid, carbopol, carboxymethylcellulose, hydroxyalkylcellulose, dextran sulfate, chitosan, xanthum gum and a water-soluble vinyl polymer, and combinations thereof.

4. The pharmaceutical composition of claim 1, wherein the L-carnosine zinc comprises a plurality of particles having an average particle size between about 0.5 micron and about 2 microns.

5. The pharmaceutical composition of claim 1, wherein the composition is a hydrogel.

6. The pharmaceutical composition of claim 1, wherein the composition has a viscosity suitable for lavage of the oropharynx.

7. The pharmaceutical composition of claim 1, wherein the composition has a viscosity suitable for swallowing.

8. The pharmaceutical composition of claim 1, wherein the composition has a viscosity suitable for swallowing.

9. A method of treating the gastrointestinal mucosa, comprising:
administering a composition comprising a plurality of L-carnosine zinc particles in an aqueous hydrogel matrix directly to a wound site, ulcer, or site of inflammation on the gastrointestinal mucosa.

10. The method of claim 9, wherein the administering comprises applying the composition via an endoscope.

11. A method of treating the oropharyngeal mucosa, comprising:
administering a composition comprising a plurality of L-carnosine zinc particles in an aqueous matrix directly to a wound site on the oropharyngeal mucosa.

12. The method of claim 11, wherein the administrating comprises:
contacting the oropharyngeal mucosa with the composition.

13. The method of claim 12, further comprising expectorating any remaining composition after contacting the oropharyngeal mucosa.

14. The method of claim 12, wherein said contacting is conducted for about 0.5 to about 5 minutes.

15. A method of treating disorders of the upper gastrointestinal tract, the method comprising:
contacting the upper gastrointestinal tract of a patient in need of such treatment with a composition comprising a plurality of L-carnosine zinc particles.

16. The method of claim 15, wherein the plurality of L-carnosine zinc particles are present in an aqueous hydrogel matrix, tablet, or capsule dosage form.

17. The method of claim 15, wherein the contacting is facilitated by swallowing the composition.

18. A method for treating a wound site in a mucosa, comprising:
causing carbonyl trapping activity of the L-carnosine moiety within intra-molecular atomic scale proximity to a zinc moiety at the site of mucopathy by administering a composition comprising a plurality of L-Carnosine zinc particles in an aqueous matrix to a patient in need of such treatment.

19. A method for enhancing gastroesophageal H. pylori eradication in the treatment of a stomach cancer patient comprising:
administering an L-carnosine zinc composition having an average particle size of about 0.1μ, to about 2μ, to a patient in need thereof.

20. The method of claim 19, wherein the L-carnosine zinc is administered as a hydrogel.

21. A pharmaceutical composition comprising:
L-carnosine zinc particles having an average particle size of about 0.1μ, to about 2μ.
